(19) United States
(12) Patent Application Publication
(10) Pub. No.: US 2003/0171741 A1
(43) Pub. Date: Sep. 11, 2003

(54) Catheters for clot removal

(75) Inventors: Robert Ziebol, Blaine, MN (US); Christopher H. Porter, Woodinville, WA (US)

Correspondence Address: Peter C. Lando, Wolf, Greenfield & Sacks, P.C., 600 Atlantic Avenue, Boston, MA, 02210, US

(73) Assignee: LaTIS, Inc., Coon Rapids, MN

(21) Appl. No.: 10/295,003

(22) Filed: Nov. 14, 2002

Related U.S. Application Data
(60) Provisional application No. 60/332,226, filed on Nov. 14, 2001.

(51) Int. Cl. A61B 18/24

(52) U.S. Cl. 606/7; 606/15

(57) ABSTRACT

Improved catheters for clot removal. A catheter-containing light guide may be passed through a clot. Light may emanating from the light guide as the light guide is passed forward through the clot and/or is drawn back through the clot in order to ablate the clot. In one set of embodiments, the invention provides for methods and systems for delivering the light guide through the clot and for drawing it back through the clot to irradiate and/or ablate the clot. The invention provides, in another set of embodiments, methods and systems to deliver the light energy or radiation to the clot to perform ablation, for example during a single pass. The invention also provides, in yet another set of embodiments, methods and systems to increase the efficiency of the ablation, for example, by increasing the spot size. In still another set of embodiments, the invention provides methods and systems to remove blood from the area between the point on the light guide where the light exits and the portion of the clot to be ablated (“clearing the field”). These and other embodiments of the invention may be combined in various ways to provide a catheter system optimally designed for the particular application.

### RELATED APPLICATION

This non-provisional application claims the benefit of U.S. Provisional Patent Application Serial No. 60,/332,226, filed Nov. 14, 2001, entitled “Improved Catheters for Clot Removal,” by Ziebol, et al., incorporated herein by reference.

### BACKGROUND

1. Field of the Invention

This invention generally relates to catheters, and, in particular, to catheters for clot removal.

2. Description of the Related Art

Clot removal catheters often need to advance the catheter over a guide wire and through the clot multiple times for effective clot removal. This requirement for multiple advancements of the catheter may be undesirable, particularly for small, fragile vessels such as those in the brain, for example, because of the added time required to perform the clot removal process and/or because of inherent safety problems associated with advancement and retraction of guide wires on such vessels.

Some catheters required to reach a clot such as a brain clot may have a small diameter, and/or may be very floppy, for example, so as to be able to navigate vessels leading to and surrounding the clot that can be tortuous or fragile, without damage to the vessels. Because of the general floppiness of these catheters, they often cannot be advanced without a guide wire; and their small diameter may result in a small spot size for energy that may exit the catheter. In some cases, the small spot size may be the reason multiple passes can be required to ablate a clot, for example completely. As used herein, “ablation” refers to the removal of the clot, for example due to light energy. Ablation may be partial or complete, and may be due to, for example, erosion, melting, evaporation, or vaporization of at least one or more substance within the clot.

Guide wires can absorb energy from forward firing catheters, and therefore often need to be removed from the catheter before firing of the catheters. Therefore, an advancement and a retraction of the guide wire may be required for each advancement of the catheter, for example, to a new firing position. Forward firing catheters may generally follow the path of least resistance, which may result in ablation of substantially the same track through the clot during each pass.

While firing into the clot from the front of the catheter may be one effective way to perform clot removal, light coming out of the end of the catheter may expose only a small area of the clot and therefore, while effective for opening a channel through the clot, the catheter may generally not be able to clear the entire clot with a single pass. Thus, for reasons such as those indicated above, the catheter may have difficulty in clearing the entire clot with multiple passes. For multiple passes, a guide wire exchange may still be required each time the catheter is moved forward through the clot. It would therefore be preferable in many cases if the catheter and light guide could be designed so as to permit the entire clot, or substantially the entire clot, to be ablated during a single pass of the catheter through the clot, either forward or backward, thereby permitting faster and more effective clot removal. It would also be desirable in some cases, to the extent multiple passes may be required, if the guide wire could remain in the catheter during firing, even for a forward firing catheter, and if effective spot size could be increased and/or if the ablation efficiency could otherwise be enhanced. This invention relates to various techniques for achieving these and other objectives.

### SUMMARY OF THE INVENTION

This invention generally relates to catheters, and, in particular, to catheters for clot removal.

In one set of embodiments, the invention includes a system for ablating a clot by passing a light guide through the clot and then drawing the light guide back through the clot. In some cases, light energy may be applied to the light guide when it is passing in at least one direction through the clot, and the light guide is at least one of forward firing and side firing.

In another set of embodiments, the invention includes a wire for use in an optical ablation system where light energy used for ablation may impinge on the guide wire. In some cases, the guide wire may be formed so that at least the distal end thereof has a low refractive index that is at least at the wavelength of the light energy.

Other advantages, novel features, and objects of the invention will become apparent from the following detailed description of non-limiting embodiments of the invention when considered in conjunction with the accompanying drawings, which are schematic and which are not intended to be drawn to scale. In the figures, each identical or nearly identical component that is illustrated in various figures typically is represented by a single numeral. For purposes of clarity, not every component is labeled in every figure, nor is every component of each embodiment of the invention shown where illustration is not necessary to allow those of ordinary skill in the art to understand the invention. In cases where the present specification and a document incorporated by reference include conflicting disclosure, the present specification shall control.

### BRIEF DESCRIPTION OF THE DRAWINGS

Non-limiting embodiments of the present invention will be described by way of example with reference to the accompanying drawings in which:

FIG. 1 illustrates various embodiments of the invention within a vessel;

FIG. 2 illustrates various embodiments of the invention having an catheter, a portion of which may be able to oscillate;

FIG. 3 illustrates various embodiments of the invention where the area of contact of the light beam may be increased;

FIG. 4 illustrates another embodiment of the invention;

FIG. 5 illustrates an embodiments of the invention having a target;

FIG. 6 illustrates an embodiments of the invention having the ability to flush a fluid;

FIG. 7 illustrates various embodiments of the invention; and

FIG. 8 illustrates an embodiments of the invention having multiple fibers.

### DETAILED DESCRIPTION

In the present invention, a catheter-containing light guide may be passed through a clot. Light may emanating from the light guide as the light guide is passed forward through the clot and/or is drawn back through the clot in order to ablate the clot. In one set of embodiments, the invention provides for methods and systems for delivering the light guide through the clot and for drawing it back through the clot to irradiate and/or ablate the clot. The invention provides, in another set of embodiments, methods and systems to deliver the light energy or radiation to the clot to perform ablation, for example during a single pass. The invention also provides, in yet another set of embodiments, methods and systems to increase the efficiency of the ablation, for example, by increasing the spot size. In still another set of embodiments, the invention provides methods and systems to remove blood from the area between the point on the light guide where the light exits and the portion of the clot to be ablated (“clearing the field”). These and other embodiments of the invention may be combined in various ways to provide a catheter system optimally designed for the particular application.

It is to be understood, as used herein, a “clot” refers to any coagulated deposit located within a vessel within the body of a subject, such as a blood vessel, a or a vessel in the brain. For example, the clot may be a blood clot, a plaque deposit, or the like. Preferably, the clot is a blood clot.

The term “patient” or “subject” is meant to include mammals such as humans, as well as non-human mammals such as non-human primates, cows, horses, pigs, sheep, goats, dogs, cats, or rodents such as mice or rats.

Light having any frequency may be used in the current invention. In one set of embodiments, the light has a frequency/wavelength such that the light is preferentially absorbed by the clot or other target site that is to be treated with the invention. In one embodiment, the light has a frequency that is absorbed preferentially by the clot or target site, compared to the surrounding vessel. In one set of embodiments, the light is absorbed by a light-absorbing substance which then treats the clot or other target site, for example, due to heating effects, shock wave creation, etc.

As used herein, a “light guide” is a guide that is able to transmit light therethrough. The light may originate at another location, for example internally or externally of the subject, and be transmitted by the light guide in the subject to a site of action, for example, a clot. In one set of embodiments, the light guide is an optical fiber. In certain cases, the light guide may include one or more fibers. For example, a light guide may include three, four, five, six, seven, or more fibers. In one set of embodiments, the light guide is substantially flexible or is otherwise able to return to its original shape after being distorted in some fashion.

In one aspect, the present invention provides various methods and systems able to reduce or eliminate multiple guide wire insertions. In one set of embodiments, the invention includes a light guide, which may be optically transparent, or translucent in some cases. The catheter may be moved back and forth with the guide wire in place, or mounted to the end of the catheter. The invention, in another set of embodiments, includes an optically clear catheter, where the catheter is generally stationary, while the light guide (e.g., an optical fiber) may be moved, for example, back and forth. In yet another set of embodiments, the invention includes a side firing system with a standard guide wire in place. The side-fired energy generally does not impinge on the guide wire. As used herein, a “side firing” system is a system that is able to direct energy away from the sides of the catheter (i.e., substantially perpendicular to the longitudinal axis of the catheter), while a “forward firing,” a “front firing” or an “end firing” system is a system that is able to direct energy substantially along the longitudinal axis of the catheter.

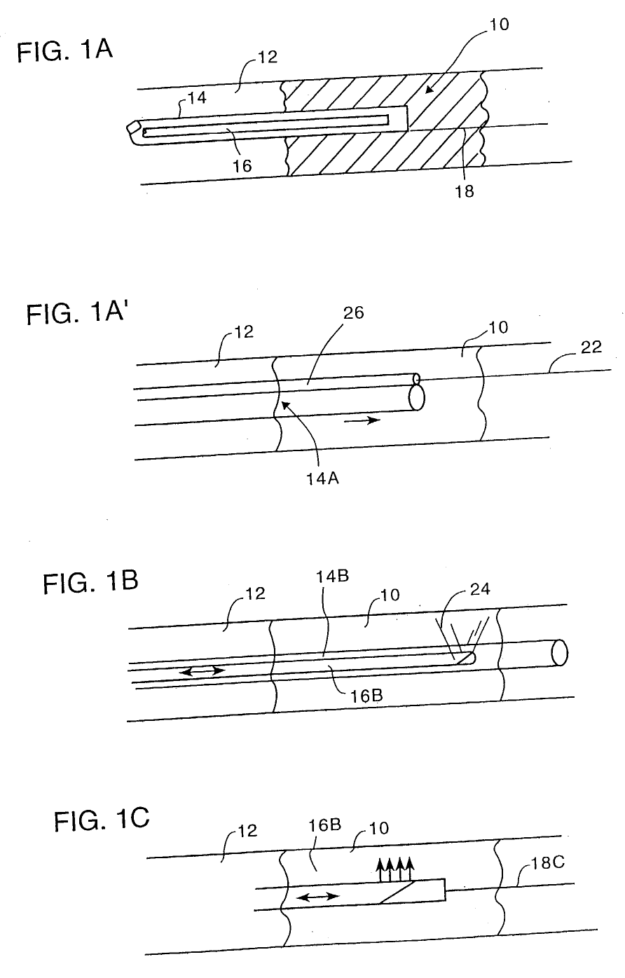

In accordance with the above, FIG. 1 illustrates examples of various mechanisms and embodiments which may be utilized for traversing a clot 10 in a vessel 12. In FIG. 1A, a catheter 14 is shown having a light guide 16 passing therethrough and having a fixed guide wire 18 extending from its distal end. For the embodiment shown in FIG. 1A where light guide 16 transmits light out the end thereof, guide wire 18 may be formed of a material which does not absorb radiation at the wavelength being emitted from light guide 16, and which may reflect substantially all such light impinging thereon. In one embodiment, the material may have a low refractive index at the emitted wavelength, in contrast with materials currently used for guide wires that do not generally have such a low refractive index. For purposes of this application, a “low refractive index” is defined to be a refractive index generally comparable to that of polytetrafluoroethylene, or lower. The embodiment of FIG. 1A has a single lumen. Guide wire 18 fixed to the end of catheter 14 may be utilized to guide the catheter through clot 10. Catheter 14 may then be pulled back through clot 10 while applying light energy through light guide 16 to ablate the clot. An optically transparent fluid may be flowed through the catheter around light guide 16 and out the end of the catheter to clear the field between the end of the light guide and the clot.

In one set of embodiments, the system includes a guide wire that has been treated in some fashion to resist degradation or damage due to light. The guide wire may be positioned within the catheter, for example, loosely or bound to a surface of the catheter, or the guide wire may be external of the catheter. In some cases, the guide wire may be positioned such that light energy reaches the guide wire; for example, the guide wire may be positioned in front of the light guide, or the guide wire may be positioned such that at least a portion of the light emanating from the sides of the catheter reaches the guide wire. In one embodiment, the guide wire may be coated with a material that resists degradation, or reflects light energy. In some cases, the guide wire may be treated with a material (e.g., a material having a low index of refraction) to cause the guide wire to become reflective due to a difference in the index of refraction.

In FIG. 1A′ the catheter 14A differs from the catheter 14 of FIG. 1A in that instead of having a fixed guide wire 18 extending from the end of the catheter, the catheter has a guide wire lumen 20 though which a guide wire 22 passes. Once guide wire 22 passes through clot 10, it may remain stationary as catheter 14A is moved over the guide wire through the clot, then pulled back through the clot. Irradiation may occur when the catheter is moved in either one or both directions. Should it become necessary to make more than one pass through the clot, in the embodiment of FIG. 1A′′, the guide wire may not need to be advanced through the clot a second time. In some cases, it may be easier for the catheter to pass through the same opening.

In one embodiment, the system includes a guide wire that remains stationary in the clot as the catheter is moved through the clot and an exchange guide wire which passes through the same lumen at the distal end of the catheter as a light guide and is exchanged with the light guide for guiding the catheter through the clot. In one embodiment, the system includes a guide wire that is not light absorbing.

FIG. 1B shows another embodiment of the invention, where catheter 14B may be transparent to the radiation 24 applied to light guide 16B. In one embodiment, light guide 16B may be a side-firing light guide having an angled facet near its distal end, which may cause, in some cases, light to exit in a selected rotational and angular direction through the walls of catheter 14B. While for the illustrative embodiments, the angle at which light 24 emanates is a right angle, this is for purposes of illustration only, and other either forwardly facing or rearwardly facing angles may be utilized for a particular application. The reflection efficiency of the most rearwardly facing angles may be increased by uptapering the fiber in the region proximal to the angle. Uptapering, which is the gradual increase in the core diameter along the length of the fiber, increases the efficiency by decreasing the effective numerical aperture (angular spread) of the light that hits the angled reflector. Catheter 14B may be formed along its entire length of a suitable transparent material, for example a suitable fluorocarbon, polyurethane, silicone, nylon, polyethelene, polyester, or other suitable transmitting materials, or only a selected length along the distal end of the catheter may be formed of such material. In certain embodiments, a selected number of openings or transparent windows may be formed in the distal end of the catheter. In some cases, the length of the transparent distal end portion may be sufficient so that the entire portion of the catheter passing through any clot 10 to be treated may be generally optically transparent.

In the embodiment of FIG. 1B, a guide wire may first be passed into vessel 12 and through clot 10. Catheter 14B may then be passed over the guide wire and through the clot. The guide wire may then be removed and the light guide 16B may be inserted in the catheter. This may be accomplished, for example, by having a double lumen to single lumen catheter, or a single lumen catheter with the guide wire being fully removed from the catheter before light guide 16B is inserted. Since catheter 14B can remain stationary in the clot during the ablation process, and light guide 16B can be moved in and out, it may be preferable in certain cases to utilize a single lumen catheter. Of course, in other embodiments, a double lumen, or a double lumen to single lumen catheter may be used. For each lateral position of light guide 16B in the catheter, the light guide may be manipulated, for example, by being torqued or rotated, such that the clot may be ablated around substantially the entire catheter rather than through just one angular orientation. Thus, in one embodiment, catheter 14B may be transparent around 360 °; in other embodiments, only a portion of catheter 14B is transparent, for example, 45, 90 °, or 180 ° of the catheter may be transparent. In certain embodiments, three or four angular orientations of the catheter may be sufficient to ablate the clot; however, in other embodiments, the light guide may be rotated to irradiate four, five, six, or more randomly chosen angular positions for each lateral position of the light guide, which may provide substantially complete ablation of clot 10 during a single pass of the light guide through the clot. In some embodiments, multiple passes (e.g., three, four, five or six passes) of the light guide may be made through the clot. In some cases, at least some of the passes may be made at a different rotational position. In the embodiment of FIG. 1B, both the guide wire and the catheter may pass through the clot 10 only once, regardless of the number of passes through the clot which may be required in order to effect the desired ablation. In some cases, this may reduce the amount of trauma.

FIG. 1C illustrates another embodiment of the invention which does not utilize a catheter, but instead attaches guide wire element 18C to the end of a side-firing light guide 16B. Since light guide 16B is a side firing light guide, guide wire 18C may not necessarily be of a non-radiation absorbing material. In operation, guide wire 1 8C may be utilized to guide light guide 16B through clot 10, and can then pulled back through the clot with the lightguide. Light guide 16B may be manipulated (e.g., torqued or rotated) as indicated for the light guide of FIG. 1B at each lateral position, for example to ablate the clot at such lateral position. In some cases, the light guide may be pulled through the clot with a single angular orientation. In certain embodiments, guide wire 18C may be used to reinsert the light guide through the clot, for instance such that the angular orientation of the light guide is changed, either before or after such reinsertion. Multiple passes (e.g., three, four, five or six passes), for instance with randomly selected different orientations may allow ablation of the clot. The embodiment of FIG. 1B may thus require fewer passes of guide wire/catheter/light guide through the clot, and may allow easier manipulation of light guide 16B in catheter 14B.

Of course, the four embodiments shown in FIG. 1 are examples only. Other embodiments are also within the scope of the present invention. For example, the embodiment of FIG. 1A may be utilized with a transparent catheter 14B and a side-firing light guide 16B. Other variations are also possible.

In another aspect, the present invention provides various methods and systems able to increase the efficiency of energy delivery in general, and more specifically, to increase the ablation or spot size. In one set of embodiments, the catheter and/or the light guide tip is moved and/or biased, for example, so as to not always be aimed in the same direction. In another set of embodiments, side firing occurs through the catheter. In yet another set of embodiments, an ablating beam, for example, an off-axis ablating beam, may be oscillated and/or rotated. The invention, in yet another series of embodiments, includes a target or target site used to improve ablation.

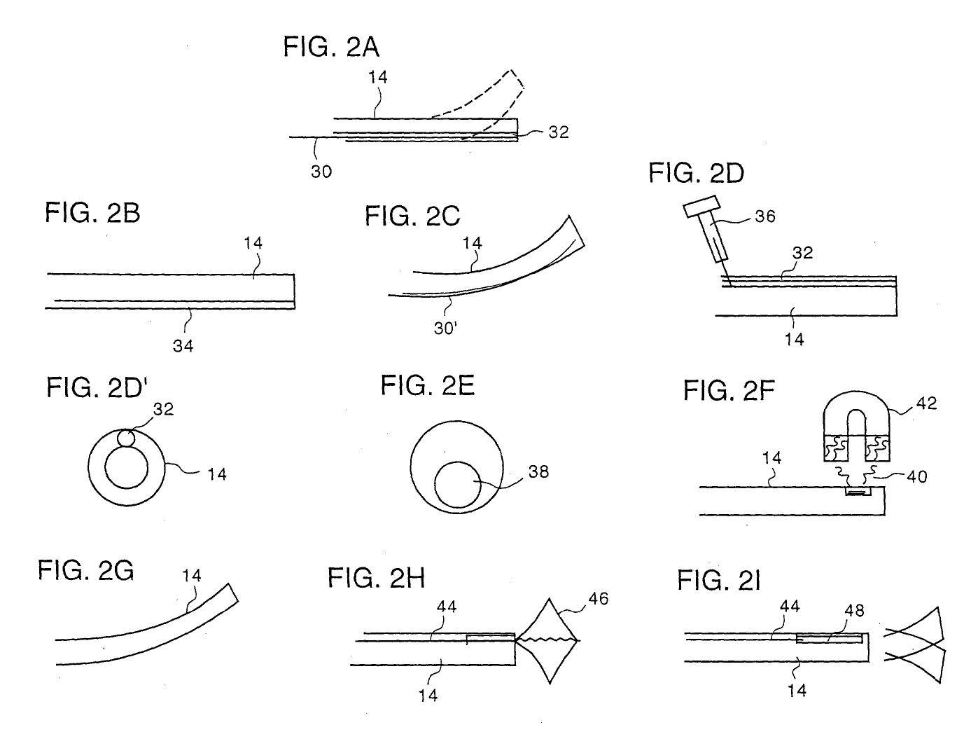

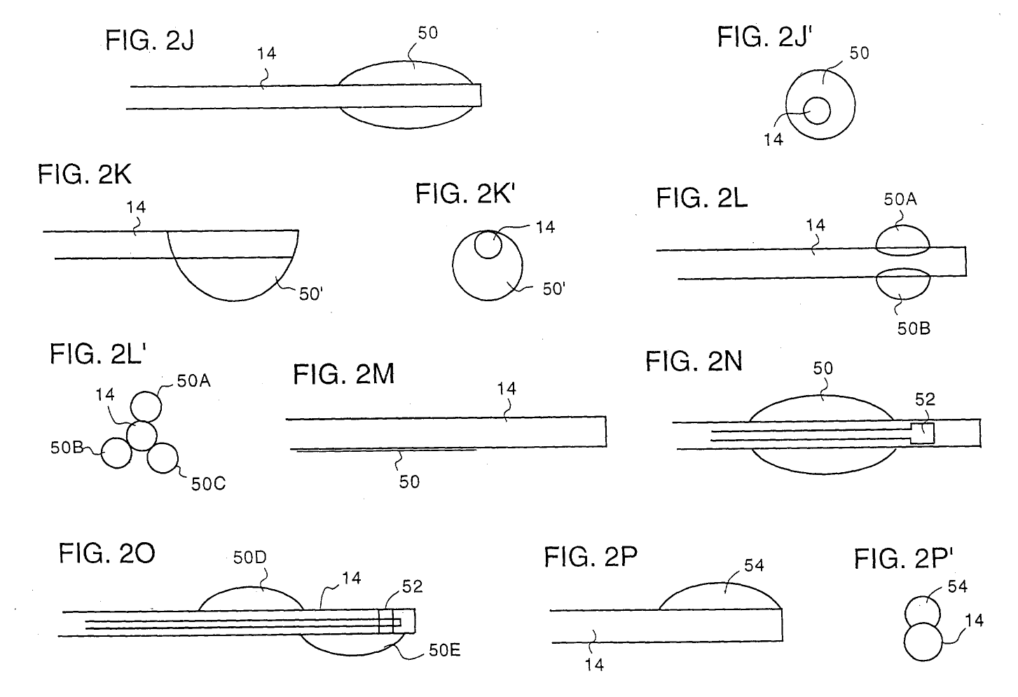

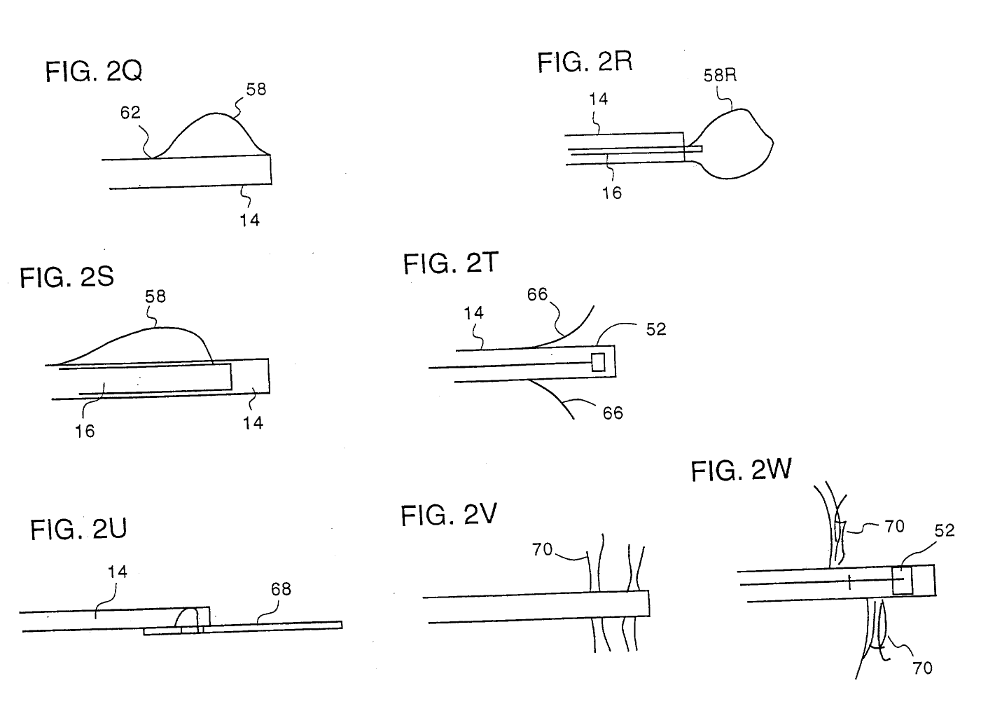

For example, in the embodiments illustrated in FIG. 1, in some cases, the radiation emanated from the tip of the catheter was used to provide a small field which generally would not be great enough to clear or ablate the entire clot. In certain cases, a side firing light guide may be used which may be manipulated to multiple random angular positions, allowing ablation of most or all of the clot. In another set of embodiments, some controlled oscillation of the distal end of the catheter and/or the light guide may be used so as to permit a wider radiation field to be obtained in a controlled manner. In some cases, this procedure may require less radiation to be applied at each lateral position of the light guide. FIGS. 2A-2W illustrate a variety of these embodiments. In some cases, the angle the distal end of the catheter may be controlled in a selected manner. In certain embodiments shown in FIGS. 2A-2W, controlled movement of the distal end of the catheter in a plane perpendicular to the walls of the vessel 12 may be possible.

In FIG. 2A the distal end of catheter 14 may be biased to point at a selected angle. A wire 30 may then be fitted in a small lumen 32 in the catheter or a wall thereof. The wire may be inserted into the distal end of the catheter to straighten the catheter, e.g., as shown in solid lines in FIG. 2A. The wire may then be retracted, for example, to permit the catheter to angle under its normal bias as shown in dotted lines. The catheter may be torqued or otherwise manipulated to change the angle at which the catheter extends and thus the firing angle. In some cases, the torque may be produced with wire 30 extended into the distal end to straighten the end. Several randomly selected angles, in some cases, may be sufficient to fully ablate a clot. In some cases, the angular position of the catheter may generally be easier to control than for the light guide.

In FIG. 2B, a temperature sensitive bimetal component may be either attached to a wall of catheter 14 or passed through a small lumen in the catheter or in the wall of the catheter. The bimetal element may extend along the entire length of the catheter or may exist only at the distal end thereof. To bias the distal end of catheter 14 to an angled position, the bimetal component may be heated either by having the bimetal element itself or a heat conducting wire attached to the bimetal element extend to the proximal end and heating the proximal end of such wire or component, or by passing a warm fluid into catheter 14 or into the lumen containing bimetal component 34. Chilling the bimetal component by applying cold to the proximal end thereof or by use of cold water may also result in the distal end of the catheter being angled. The temperature to which the bimetal element 34 is heated or cooled may determine the degree to which the distal end of the catheter is angled. The embodiment of FIG. 2B otherwise operates in substantially the same manner as the embodiment of FIG. 2A.

For FIG. 2C, instead of catheter 14 being biased and wire 30 being straight, catheter 14 is straight and wire 30′ is biased. Angling of the end of the catheter may be controlled, for example, by inserting the wire into a channel or lumen formed in the catheter. To cover the entire clot, either the catheter 14 may be rotated as for the prior embodiments or bias wire 30′ may be rotated to change the direction in which the distal end of the catheter points.

The embodiment of FIGS. 2D and 2D′ is similar to that of FIG. 2A in that the distal end of the catheter is normally biased and a channel 32 is formed in one wall of the catheter. However, for this embodiment of the invention, a fluid may be injected into channel 32 by for example a syringe 36 to straighten the catheter, rather than using wire 30. The fluid pressure applied by syringe 36, in some cases, may be used to determine an angle of the catheter.

FIG. 2E is similar to FIG. 2D, except that pneumatic or hydraulic pressure may be applied to a lumen 38 in the catheter to straighten the catheter rather than to a channel 32 in the wall of the catheter. Similarly, FIG. 2G illustrates the use of a biased catheter 14 which is not rigidified as for the prior embodiments, and may be torqued while in its biased condition.

FIG. 2F shows an embodiment wherein a slug or ring 40 of a ferrous material may be mounted on or attached to the distal end of catheter 14 and a magnet 42 outside the patient's body may be used to control the angular position of the tip. The magnet may, for example, be moved over the patient to achieve desired angular positions, for example, over the head, chest, legs, or other areas where the catheter is located.

FIGS. 2H and 2I illustrate various embodiments wherein a rotatable wire having a screw thread at its distal end may be used to control catheter angle/position. For FIG. 2H, the rotating wire 44 may end in a jackscrew which can expand or contract a basket 46 that interacts with the walls of vessel 12. By utilizing an eccentric basket 46, the position and/or angle of the distal end of catheter 14 may be controlled. Similarly, in FIG. 2I, rotation of wire 44 may cause a screw 46 to be pulled or pushed. Screw 46 may be attached off-center to catheter 14. In some cases, pulling or pushing of the screw may raise or lower the angle of the distal end of the catheter, which may permitt the catheter to scan a swath of selected width through the clot.

FIGS. 2J and 2K respectively illustrate a single balloon and a double balloon embodiment for controlling the position and/or angle of the distal end of catheter 14. In FIGS. 2J and 2J′, a balloon 50 may surround the distal end of catheter 14 and, when inflated, may interact with the walls of vessel 12, for example, to control the position in the vessel of the distal end of the catheter. In some cases, catheter 14 may be eccentrically mounted in balloon 50 as shown in FIG. 2J′. FIGS. 2K and 2K′ illustrate another form of eccentric mounting of catheter 14 in a balloon 50′ which may result in controlled movement of the distal end of the catheter to ablate at least a portion of the clot. FIGS. 2L and 2L′ illustrate an embodiment wherein three balloons 50A-50C may be mounted around the periphery of catheter 14 at its distal end. Balloons 50A-50C may be individually blown up or blown up in various combinations to move the distal end of catheter 14 across substantially the entire clot 10 in a controlled manner, in some cases while continuing to apply irradiation.

FIG. 2M illustrates a non-symmetric balloon 50 in the wall of catheter 14 or in a lumen of the catheter. FIG. 2N illustrates an embodiment wherein an optically transparent flush fluid used to clear the field may be used, for example under control of a restrictor or a valve 52, to inflate balloon 50. A restrictor or valve may also used for the embodiment of FIG. 2M. FIG. 2O is similar to FIG. 2N, except that balloons 50D and 50E may be sequentially positioned along catheter 14 and may, in some cases, be sequentially inflated under control of restrictor or valve 52 to permit scanning of the distal end of catheter 14. FIG. 2P illustrates the use of an eccentric catheter having a projection 54 affixed to the distal end thereof, which may control the position of the distal end of the catheter in the vessel. The catheter may be scanned, for example, by torquing or otherwise manipulating the proximal end thereof.

FIGS. 2Q-2S illustrate various embodiments wherein a protruding wire may be utilized, for instance in much the same way as a balloon is utilized for some of the prior embodiments. In particular, in FIG. 2Q a wire 58 may be extended along an outer wall of catheter 14. In certain cases, the wire may be secured at a point 60 near the distal end of the catheter. In some embodiments, the wire may be laterally slidable in an eyelet 62 proximally spaced from point 60. The wire may be pushed from the proximal end to extend as shown in the figure to move the distal end of catheter 14. The degree to which the wire is extended may determine the degree of movement. FIG. 2R illustrates an embodiment wherein a wire basket 58R may be attached to the end of light guide 16. In some cases, the basket may extend to interact with the walls of vessel 12 as the light guide is pushed out of catheter 14. In some cases where basket 58R is symmetric, the basket may be served as a centering device to move light guide 16 and/or catheter 14 off the bottom of the vessel. In contrast, in cases where the basket is eccentric, the basket may be used in one or manners as previously indicated to control the lateral position of the light guide and/or catheter. The amount by which the light guide protrudes from the catheter may control the extent of basket 58R, and thus, in some cases, may control the point on the clot being irradiated. FIG. 2S shows an embodiment of the invention wherein wire 58 normally rests inside catheter 14 and may be pushed out of the catheter as light guide 16 is inserted therein.

In another set of embodiments, the wire may interact with the walls of vessel 12 to control the position of the distal end of catheter 14. For instance, FIG. 2T illustrates an embodiment of the invention where fluid pressure, for example from the optically transparent fluid used to clear the field, may open or extend one or more flaps 66 which can interact with the walls of the vessel, for example, to control the distal position of catheter 14. Flaps 66 may either be symmetrically positioned to center the catheter or eccentrically positioned to achieve off-center positioning of the catheter. FIG. 2U shows an eccentric tip 68 that can be attached to the distal end of catheter 14, which tip may or may not be a guide wire. Catheter 14 may be steerable in this embodiment, for example, by rotating the catheter about eccentric tip 68.

FIGS. 2V and 2W illustrate embodiments where fluid jets may be utilized to control catheter position. For example, in FIG. 2V, fluid jets, which may be, for example, jets of clearing fluid, may pass through openings in the catheter wall and may be substantially uniformly distributed in some cases. In FIG. 2W, a restrictor or valve 52 may be used to permit sequential operation of the jets 70, for instance, to permit eccentric positioning of the catheter within the vessel.

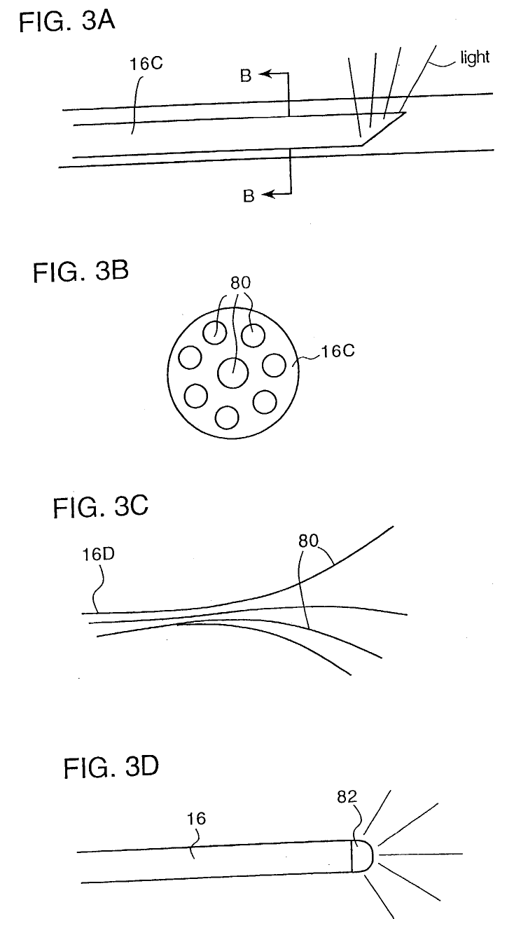

In another aspect of the invention, as illustrated in FIG. 3, the invention includes various ways in which the area of contact of the light beam on the clot 10 may be increased. The energy applied to the light guide may be increased to provide a larger spot, for example, so that the energy density applied to the clot may remain above the energy threshold required for clot ablation.

FIG. 3A illustrates a side-firing light guide either inside the vessel or inside a catheter 14 in certain cases. FIG. 3B is a cross-section through the fiber 16C of FIG. 3A showing that this fiber may be formed of seven individual fibers 80. Of course, in other embodiments, there may me more of fewer numbers of individual fibers therein. In some cases, the seven individual fibers may more flexible than a single larger fiber having the same light carrying capacity. The pattern for the fibers shown in FIG. 3B has seven fibers wrapped around a central core fiber; however, other fiber bundle patterns may be utilized. Light may be applied to all of the fibers 80 simultaneously or may be applied to them either individually or in groups in some predetermined pattern. The total energy applied may be greater than with an individual fiber in certain cases. The fiber bundle 16C may also be utilized to deliver light from the end of the light guide rather than side firing, or the catheter may include a combination of end and side firing.

In one aspect, the side-firing light guide directs energy using a reflective surface, for example, a mirror, or a difference indexes of refraction in that causes reflection to occur. For example, the reflective surface may be a dielectric mirror, an air gap, a silvered surface, etc. The differences in indexes of refraction may be at the junction of two solid materials, a solid material and a liquid, two liquid materials, etc.

FIG. 3C shows a fiber bundle 16D being used as the light guide, with the fibers being spread at their distal end by a selected amount to increase the area of light contact. The amount by which the fibers are spread may determine the area of contact. In the embodiment of FIG. 3B, the fibers 80 may be energized simultaneously or may be energized individually or in groups in some predetermined pattern. FIG. 3D illustrates still another embodiment of the invention, where light guide 16, which may be formed of a single fiber or of a fiber bundle as shown, for example in FIG. 3B, may have a lens 82 mounted at its distal end. The lens may disperse light from the fibers in any desired pattern, for example, over a larger area of contact. The lens may be located, for example, on the distal end of the catheter, the light guide, or a fiber within the light guide.

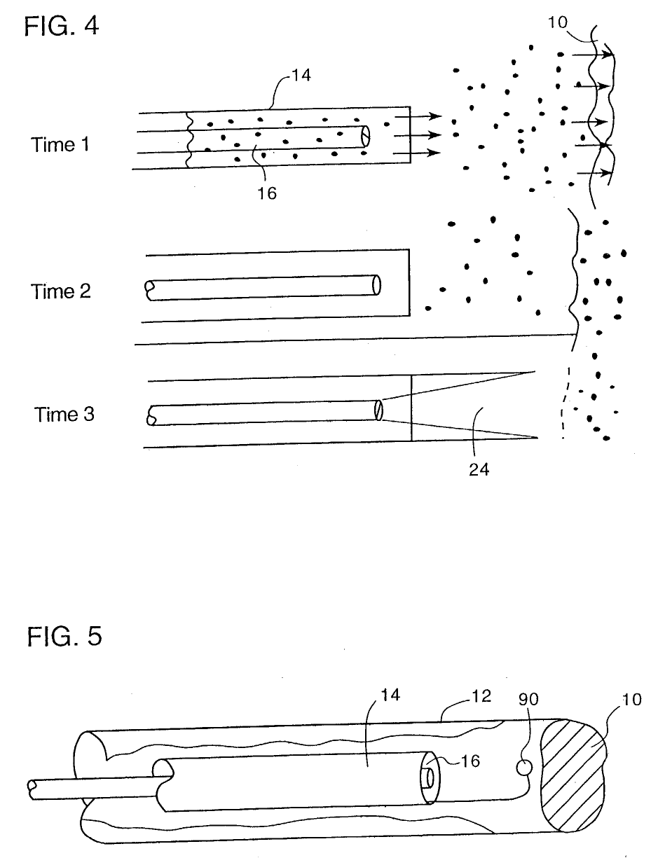

FIG. 4 and FIG. 5 illustrate techniques for using a “target” to facilitate the use of light energy to clear a clot, for example, a clot in a brain vessel. Techniques involving targets may be particularly useful where the clot is a piece of material which has become lodged in the vessel, as opposed to a clot which has grown at the site, and may thus not be of a material which absorbs the optical radiation normally used to ablate clots, or for treating plaque, calcified material or other material not containing a useful chromophore which may be formed in the vessel. However, even a blood-containing clot at the site may not be an optical absorber for the applied radiation. In some cases, the target may be used to treat any deposit located within the vessel. For example, the deposit may be a deposit that does not substantially absorb the incoming light (e.g., a transparent deposit).

Referring to FIG. 4, at time 1, a fluid bolus of a highly absorbent material, for example carbon, may be sent down catheter 14. Light guide 16 may or may not be in the catheter during this portion of the procedure. This material may diffuse into, coat, or otherwise affect clot 10. At time 2, the light-absorbent material diffuses into and is absorbed into the walls of the clot. A clear fluid or solution (e.g., saline, blood, plasma, a contrast fluid such as an X-ray contrast fluid, or the like) may then passed through the catheter to clear the field and, at time 3, once the field is cleared, the light energy 24 may be delivered to the clot enhanced with the absorbent material. The absorbent material may diffuse into and/or becoming part of the clot may absorb more of the light energy than would be absorbed by the clot alone, which may cause greater heating and/or ablation of the clot. In some cases, the absorbing material may also be heated to a point where they explode causing shock waves.

In FIG. 5, a target 90 may be mounted in the path of light emitted from light guide 16. Target 90 may be formed of a material which may be highly absorbent of the light energy emitted from the light guide. The light energy may affect target 90, for example, by superheating target 90. In some cases, a vapor bubble may be formed. When this bubble collapses, it causes a shock wave containing substantial energy which is operative to effect ablation. In certain cases, these effects may occur in addition to the direct impinging/ablation of clot 10.

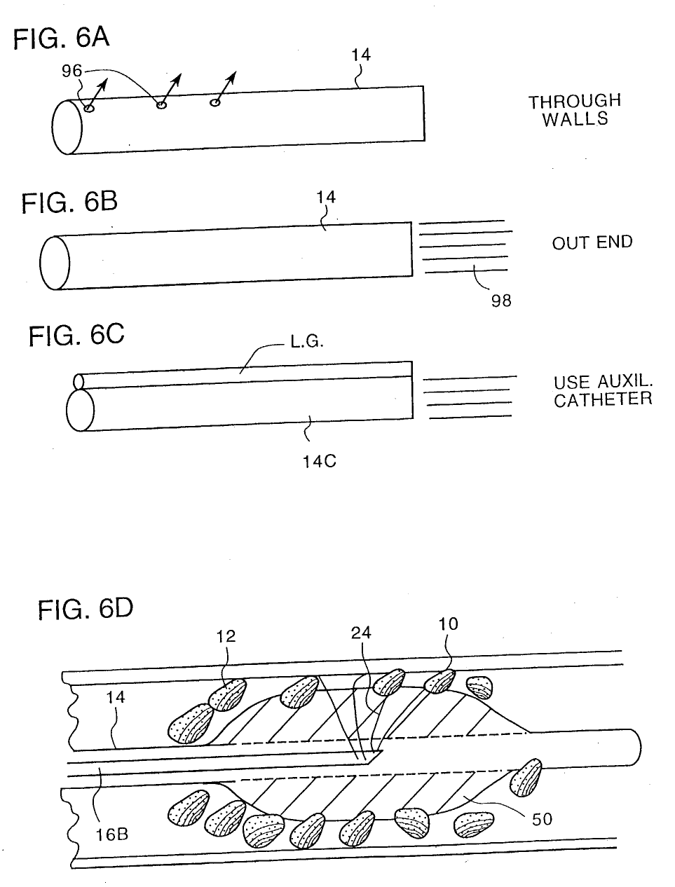

In another aspect, the invention includes systems and methods for clearing the field. For example, FIG. 6 illustrates various techniques which may be utilized for clearing the field. FIGS. 6A-6C show various techniques for utilizing a fluid flush for field clearing, while FIG. 6D illustrates a technique for pushing the blood out from between catheter 14 and the walls of vessel 12. FIG. 6A illustrates a catheter 14 for use with a side-firing light guide 1D (FIG. 1B) where holes 96 may be provided in the wall of the catheter through which the clearing or flush fluid may flow. Holes 96 may be replaced by slits, a single large opening or any other suitable opening through which the clear flush fluid may flow. FIG. 6B illustrates a procedure where flush fluid 98 flows out the distal end of the catheter, while FIG. 6C illustrates a two-lumen catheter 14C wherein the light guide 16 is in a smaller upper lumen and flush fluid flows through a larger lower lumen. Other techniques known in the art for delivering a clear flush fluid to the field between the light guide and the clot may also be utilized.

In FIG. 6D, the field may be cleared by using a fluid-filled balloon 50. The fluid may be air or another gas or a liquid, for example, pumped through catheter 14 to expand and press the balloon against the walls of clot 10 or of vessel 12, for example, to squeeze blood, blood clots, or other contaminants out from between the light 24 emanating from side firing light guide 16b and clot 10. The walls of catheter 14 may, in some cases, be sufficiently porous and/or have holes formed therein in the area adjacent balloon 50 to facilitate the filling thereof. Other techniques known in the art for clearing the field may also be utilized.

In some embodiments, the catheter in contact with the walls of the clot may be sufficient to clear the field so that techniques such as those shown in FIGS. 6A or 6D would not be required.

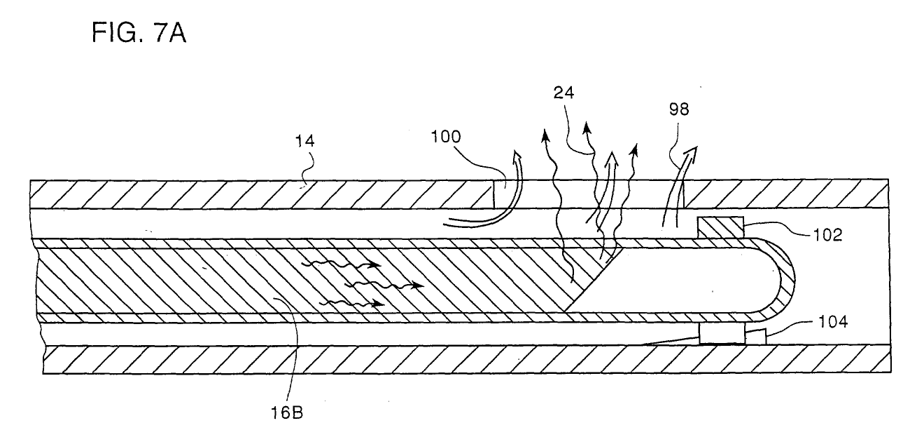

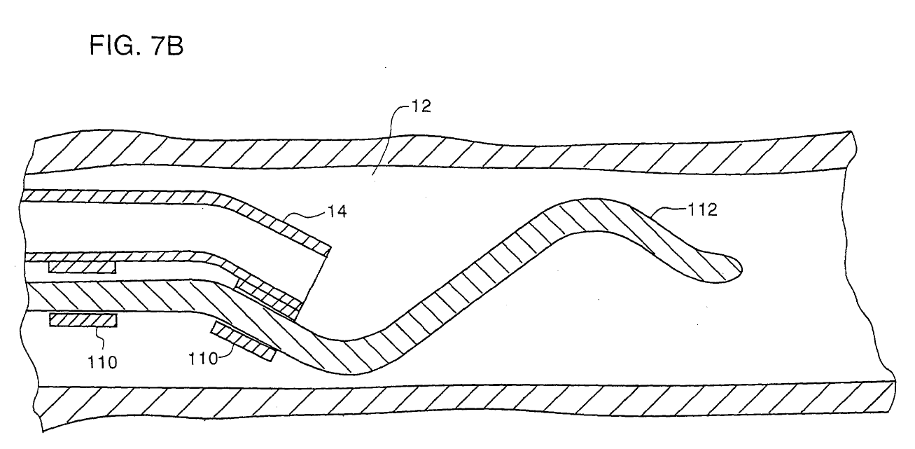

FIGS. 7A-7B illustrate additional embodiments of the invention. Referring first to FIG. 7A, catheter 14 may include an opening in its walls, for example, one or a plurality of holes 96 (FIG. 6A), or a single opening 100, through which, in one set of embodiments flush or clearing fluid 98 may flow. In some embodiments, side-firing light guide 16B may transmit light energy 24 through opening 100. Light guide 16B may have at least one projection 102 formed at its distal end which, when the light guide is inserted into the catheter, may fit into a channel formed in guide 104. Guide 104 may, in some cases, be longer than is shown in the figure and the channel would be wider at its proximal end than at its distal end. In some embodiments, guide 104 may spiral as it narrows, for example, so that projection 102 may enter the channel and be guided by the channel to control the orientation of fiber 16B. The end of the channel in guide 104 may also serve as a stop, for instance, to assure that the fiber bevel for side firing was properly positioned adjacent opening 100. Projections 102 may, in certain embodiments of the invention, restrict fluid flow beyond the projections, for example, so as to maximize the flow of clearing fluid through opening 100. In some cases, the end of catheter 14 may be left open to permit a guide wire to pass therethrough.

In one set of embodiments, with a spiral guide channel in guide 104 co-acting with projections 102, the rotational orientation of side-firing waveguide 16B, or in other words the direction in which the waveguide fires, may be controlled by controlling the lateral position of the light guide relative to the catheter. Opening 100 may, in this case, be in the form of a series of slits or holes 96 in an otherwise optically transparent catheter, for instance, so as to maintain the structural integrity of the catheter. A ratcheting mechanism may be employed, in some cases, to permit the light guide to have a different orientation in catheter 14 each time it is pulled back slightly and then pushed forward, thereby permitting the entire clot to be covered with, for example, any number of irradiations per lateral position, for example, three or four irradiations.

In FIG. 7B, catheter 14 has one or more railed components 110 affixed to its outer wall through which a guide wire 112 passes on a single track. In some cases, guide wire 112 may be corrugated at its distal end; in other cases, guide 112 may be relatively straight. Corrugated guide wire 112 may be rotated to control the direction in which the end of catheter 14 faces, and thus the direction in which radiation is applied. The direction in which catheter 14 faces may also be controlled by moving the catheter relative to the guide wire, or by controlling the lateral position of the catheter relative to the guide wire. This may be accomplished, for example, by either moving the catheter or the guide wire. This is another way of translating linear motion of a catheter or guide wire into a direction of orientation for the tip of the catheter. Wire 112 may be stiffer than normal guide wires in some cases. In certain cases, the tip or distal end of the guide wire may be flexible to facilitate the guide wire function. Energy losses as a result of wire 112 absorbing radiation may be reduced, in some cases, by forming wire 112, or at least the tip thereof, of a non-absorbing reflective material and/or by adjusting the position of the guide wire relative to the catheter to minimize impingement of radiation on the wire.

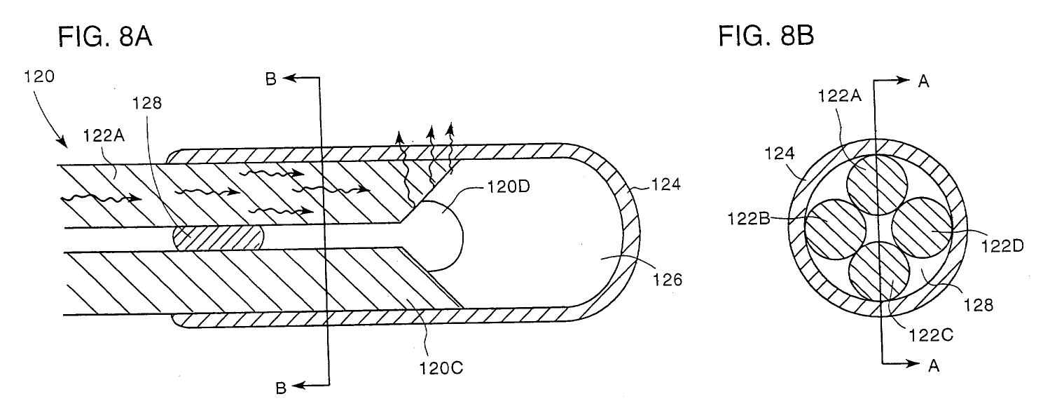

FIGS. 8A and B illustrate another embodiment of the invention. In this embodiment of the invention, light guide 120 may be formed of at least three fibers, with four fibers being shown for the illustrative embodiment of the figures. In other embodiments, light guide 120 may have five, six or more fibers. As shown in the figures, each of the fibers 122A-122D is-side-firing with its beveled end angled so that each fiber 122 fires in a different direction. In other embodiments, though, the fibers may also be end-firing, or include a combination of side and end-firing fibers. The number of fibers and the angles for each, in this figure, are selected such that the fibers combine to cover substantially an entire 360° field for preferred embodiments. Thus, this embodiment of the invention permits the entire 360° field for the clot to be covered without requiring rotation or other manipulation of the fiber or the catheter. In other embodiments, the fibers may not cover 36020 , for example, the fibers may cover a smaller area, such as a 30°, a 60°, or a 90° area, for instance in embodiments where a smaller degree of coverage is needed. Cap 124, which may be formed of an optically transparent material in some cases, for example silica, may be fused or otherwise sealingly secured to the fibers. Air space 126 may be formed between the ends of the fibers and the cap. Sealant 128 may be provided in the interstices between the fibers to facilitate maintenance of air space 126. In some cases, sealant 128 may prevent leakage of flush fluid or other liquid therein. The air space may, in certain cases provide a reflective index mismatch which, results in optimal light reflection of the fibers. Air gap 126 may permit 80%, 90%, 95%, or substantially 100% reflection. Cap 124 may also stabilizes and protects fibers 122 in certain cases.

Light guide 120 may be utilized in catheter 14 that contains a fluid therein which may be used, for example, to clear the field. The catheter may have, for example a series of slits 100 or holes 96 (FIG. 6A) formed around the periphery of a catheter which, in some cases, may be formed of material transparent to the light beam emitted from the light guide. In certain embodiments, at least at the distal portion of the catheter includes holes 96. Light may also, in some cases, be applied to the fibers 122A-122D simultaneously, or the light may be applied to all of the fibers, to the fibers individually, or to selected groups, for example, in some predetermined order.

While the invention has been particularly shown and described above with respect to a large number of embodiments, it is to be understood that these embodiments have been presented for purposes of illustration only and that features of these embodiments may be combined in a number of different ways, including the specific ways indicated, or may be used in catheters having features not specifically disclosed. The optical radiation applied to the light guides may be coherent radiation of a selected wavelength, or may be incoherent radiation, for example from a flashlamp or other lamp, which may be filtered to provide a selected band or bands of optical radiation. Thus, while the invention has been particularly shown and described above with respect to various illustrative embodiments and illustrative embodiment features, the foregoing and other changes in form and detail might be made in this invention by one skilled in the art while still remaining within the spirit and scope of the invention which is to be defined only by the appended claims.

What is claimed is:

1. A system for ablating a clot by passing a light guide through the clot and then drawing the light guide back through the clot, light energy being applied to the light guide when it is passing in at least one direction through the clot, wherein the light guide is at least one of forward firing and side firing.

2. A system as claimed in claim 1, wherein the light guide is side firing.

3. A system as claimed in claim 1, wherein a guide wire is affixed to the end of the light guide to facilitate passing the light guide through the clot.

4. A system as claimed in claim 1, wherein the light guide is mounted in a catheter which is also passed through the clot but remains stationary in the clot while the light guide is moved to ablate the clot, the catheter being optically transparent.

5. A system as claimed in claim 4, including passing a flush fluid through the catheter, the catheter having openings therein adjacent portions of the light guide from which light is to emanate.

6. A system as claimed in claim 4, wherein said light guide is formed of at least three optical fibers angled to deliver light in different directions.

7. A system as claimed in claim 6, wherein the distal end of said fibers are covered with an optically transparent cap sealed to the fibers to form an air gap between the distal end of the fibers and the cap.

8. A system as claimed in claim 4, including a mechanism for guiding the position of the light guide to a desired rotational position.

9. A system as claimed in claim 4, wherein said light guide is formed of a plurality of fibers angled to all direct light in the same direction, whereby the energy delivered by the light guide in the area over which the energy may be delivered is increased.

10. A system as claimed in claim 1, wherein said light guide is forward firing, and including a catheter through which said light guide passes and which is pulled back through the clot with the catheter, the catheter being guided through the clot by one of a guide wire fixed to the distal end of the catheter, and a guide wire passing through a separate lumen of the catheter.

11. A system as claimed in claim 1, wherein said light guide is forward firing, including a mechanism for moving the distal end of the catheter relative to the clot to facilitate clot ablation.

12. A system as claimed in claim 11, wherein said mechanism for moving the distal end of the catheter includes a mechanism for bending the distal end of the catheter to change the direction in which the light beam is directed.

13. A system as claimed in claim 1 1, wherein said mechanism for controlling the direction of the distal end of the catheter includes a mechanism for providing lateral movement of the catheter in the vessel to facilitate ablation of the clot.

14. A system as claimed in claim 13, wherein said mechanism for affording lateral movement of the distal end of the catheter includes one of an inflatable balloon mechanism, an extending wire structure, and means for extending fluid jets from the catheter, each of which interacts with the walls of the vessel to selectively position the catheter therein.

15. A system as claimed in claim 13, wherein said catheter is eccentrically mounted in the vessel and is rotatable to translate the distal end of the catheter in the vessel.

16. A system as claimed in claim 1, wherein said light guide is front firing and including a lens at the distal end of one of said catheter and a fiber.

17. A system as claimed in claim 1, wherein said light guide is front firing and including a target for enhancing the ablation by light energy.

18. A system as claimed in claim 17, wherein said target includes a light absorbing material diffused and absorbed into the clot prior to irradiation by light from said light guide

19. A system as claimed in claim 17, wherein said target includes a light absorbing material positioned in front of said clot which is heated by the light from said light guide to cause a shock wave which effects ablation.

20. A system as claimed in claim 1, wherein said system is used to ablate clots in brain vessels.

21. A system as claimed in claim 1, wherein said light guide is forward firing, and including a guide wire which facilitates movement of the light guide and/or a catheter mounting the light guide in at least one direction through the clot and which remains in place when light energy is applied to the light guide, the guide wire having at least a distal end with a low refractive index, at least at the wavelength of the light energy.

22. A guide wire for use in an optical ablation system where light energy used for ablation may impinge on the guide wire, the guide wire being formed so that at least the distal end thereof has a low refractive index at least at the wavelength of the light energy.

23. A guide wire as claimed in claim 22, wherein said guide wire is formed of a core of guide wire material having at least the distal end thereof coated with a material having said low refractive index.
